(19) United States
(12) Patent Application Publication
(10) Pub. No.: US 2019/0192878 A1
(43) Pub. Date: Jun. 27, 2019

(54) Illumination Device, Control Unit for an Illumination Device and Method for Operating an Illumination Device

(71) Applicant: LEDVANCE GmbH, Garching bei Munchen (DE)

(72) Inventor: Dieter LANG, Bruckmul (DE)

(21) Appl. No.: 16/225,946

(22) Filed: Dec. 19, 2018

(30) Foreign Application Priority Data
Dec. 21, 2017 (DE) 102017130864.4

(51) Int. Cl. A61N 5/06; H05B 37/02; H05B 33/08

(52) U.S. Cl. CPC A61N 5/0618; H05B 37/0272; A61N 2005/0663; H05B 33/0851; A61N 2005/0652; H05B 33/0863

(57) ABSTRACT

An illumination device having a first lighting unit that emits a first light in a first emission direction and a second lighting unit that emits a second light in a second emission direction. The first emission direction and the second emission direction are adjustable in such a way and/or extend in such a way relative to one another and the illumination device can be installed and/or can be positioned in such a way that in operation of the illumination device the first emission direction extends through a working plane of a user and the second emission direction extends through a facial plane of the user.

### CROSS-REFERENCE TO RELATED APPLICATIONS AND PRIORITY

This patent application claims priority from German Patent Application No. 102017130864.4 filed Dec. 21, 2017, which is herein incorporated by reference in its entirety.

### TECHNICAL FIELD

The present invention relates to an illumination device, a control unit for an illumination device and a method for operating an illumination device.

### BACKGROUND

In the last few decades mechanisms of the biological effect of light have been identified. Accordingly, the incidence of light in the blue-green spectral range causes an excitation of the protein melanopsin—a photopigment—in the retinal ganglion cells in the human eye. This protein controls so-called non-visual effects of light, including the suppression of the hormone melatonin, and the release thereof through the pineal gland and thereby has a direct influence on the control of the day-night rhythm and thus on the quality of sleep and the internal clock. Further important effects are the activation of specific areas in the brain which are responsible for activity and efficiency. The DIN SPEC 5031-100:2015-08 defines terms, values and evaluation methods for this non-visual effect of light in detail. The effects which are based on the excitation of the photopigment melanopsin are referred to by the English-language term “melanopic”.

The portions of the light radiation which are responsible for non-visual light effects are specified as a radiation measurement (Xₘₑₗ) evaluated by the melanopic action spectrum (smel(λ)). The melanopic action spectrum has its maximum in a wavelength range of at least 480 nm and at most 500 nm, in particular at approximately 490 nm, and has a value above 10% of the maximum value only between 418 nm and 562 nm. Thus the melanopic light effect is very low outside this wavelength range.

On the basis of this non-visual, biological activity of light it is desirable, in order to support the natural day-night rhythm, for living creatures, in particular humans and/or animals, to be exposed in the daytime to light which tends towards a higher melanopic action, similar to daylight. On the other hand, late in the evening, when such light can lead to difficulties in falling asleep, light with a low melanopic action is advantageous.

Especially in the area of a workplace, illumination which is dependent upon the time of day and/or adapted to activity can provide advantages. However, previously available illumination devices which achieve a melanopic effect generally illuminate a complete room or larger areas of a room. Accordingly disadvantages result due to high acquisition and installation costs and/or due to an increased energy demand. However, reduced costs would also enable an operation in a domestic area, for example in a private workplace.

Previously available lighting devices which illuminate individual workplaces are generally only oriented towards the illumination of the working plane. In particular, in operation such illumination devices only emit one type of light. The melanopic effect of such illumination systems is generally low, since only small proportions of the light from the working plane enter the user's eye by means of back reflection. Moreover, in such illumination devices the light sources for the illumination of the working plane and the illumination of the facial plane of the user are not separated with regard to their operation and with regard to their visual and non-visual effects. Accordingly both effects cannot be optimized independently of one another.

### SUMMARY

Starting from the previously described technical background, it is an object of the invention to provide an improved illumination device, by which a melanopic effect adapted to the time of day can be provided, wherein at the same time a good illumination of the visual task area is guaranteed largely independently of the melanopic effect. Furthermore, a control unit for an improved illumination device as well as a method for operating an improved illumination device is provided.

These objects are achieved by an illumination device, a control unit and a method with the features of the independent claims. Advantageous further embodiments are apparent from the subordinate claims, the description, the drawings and also the exemplary embodiments described in connection with the drawings.

Accordingly, an illumination device is specified. The illumination device comprises a first lighting unit, which in operation emits a first light in a first emission direction, and a second lighting unit, which in operation emits a second light in a second emission direction. In a particularly preferred embodiment of the illumination device the first emission direction and the second emission direction are adjustable and/or extend in such a way relative to one another and the illumination device can be installed and/or can be positioned in such a way that in operation of the illumination device the first emission direction extends through a working plane of a user and the second emission direction extends through a facial plane of the user.

The illumination device can in particular be a positionable table lamp and/or an installable ceiling light. A standard lamp which is placed alongside the work space is also conceivable. In the installed or positioned state the first emission direction can extend through the working plane and the second emission direction can extend through the facial plane of a user. In this case and in the following, the fact that a direction “extends” through a plane can mean that the direction and the plane enclose an angle of at least 60°, preferably at least 80°, and at most 120°, preferably at most 100°. Alternatively or in addition, it is possible that the first emission direction or the second emission direction are adjustable in such a way that in the installed or positioned state the illumination device extends through the working plane or the facial plane of the user.

The first lighting unit and the second lighting unit have in particular different illumination tasks. Thus the first lighting unit can be provided for illumination of the working plane. The working plane is for example a working area, such as a (writing) desk, of the user. The working plane can have a visual task area of the user, such as for example a sheet of paper or a book. The second lighting unit can be provided for illumination of the facial plane. The facial plane extends in particular along and/or through the face of a user.

It is preferably provided that the first lighting unit has a visual light effect, that is to say it is in particular configured for illumination and the second lighting unit has a non-visual light effect which supports the biological system of the user. The non-visual light effect is preferably achieved by means of a planar emission of the second light from the second lighting unit in the direction of the facial plane of the user. This can correspond to the indirect illumination by means of a spatial effect. In other words, the second lighting unit in particular emits in such a way that the second light corresponds to an indirect illumination of the facial plane via the room.

If in this application an indefinite article, such as for example “a”, “an”, etc., is used, both the singular and also the plural is meant, in the sense of “at least one” or “one at least”, etc., if this is not explicitly precluded, for example by the use of “precisely one” or “a single one”. If in the following a feature of the illumination device, in particular the numbering of a component of the illumination device (that is to say for example “first” and “second”, within a sentence is placed in brackets, moreover, this should always be interpreted in such a way that what is written within the sentence applies either for the combination of all features outside the brackets and/or for the combination of all features in the brackets. Accordingly, for example, the sentence “the first (second) lighting unit in operation emits a first (second) light” means “the first lighting unit in operation emits a first light and/or the second lighting unit in operation emits a second light”, but not “the first lighting unit in operation emits a second light” or “the second lighting unit in operation emits a first light”.

The first (second) light is in particular white mixed light. The first (second) lighting unit preferably contains a light-emitting diode (LED), in particular a plurality of LEDs, and a wavelength conversion element. It is possible that the LED and/or the wavelength conversion element of the first lighting unit differs from the LED and/or the wavelength conversion element of the second lighting unit, for example in the spectral range of the emitted light and/or of the converted light. The first lighting unit and the second lighting unit are preferably connected to a common power supply. The first light preferably has a lower color temperature than the second light. For example, a first (second) color temperature of the first (second) light is 4000 K (6500 K). In this application a color temperature is in particular the most similar color temperature (correlated color temperature, CCT).

The first (second) emission direction is in particular a main emission direction of the first (second) lighting unit. In other words, an emission profile of the first (second) lighting unit preferably has a local or global maximum along the first (second) emission direction. Alternatively or in addition, the first (second) emission direction can be perpendicular to a central point of a first (second) light exit surface of the first (second) lighting unit. In the case of a Lambertian emission characteristic the first (second) emission direction can in particular be the surface normal to the first (second) light exit surface of the first (second) lighting unit.

The first (second) lighting unit can be of flat construction. The first (second) light can then be emitted to and/or through the first (second) light exit surface of the first (second) lighting unit. In the event of an emission on the first (second) light exit surface the first (second) light exit surface is in particular designed to be reflecting and arranged in such a way that the light emitted by the first (second) lighting unit light on the first (second) light exit surface is reflected as first (second) light in the direction of the working plane (facial plane). In the event of an emission through the first (second) light exit surface, this surface is preferably at least partially light-permeable, for example transparent, translucent and/or light-scattering.

The first (second) light exit surface preferably has a size of at least 20 cm². Thus the first (second) light exit surface is in particular larger than the light exit surface of an individual light-emitting diode. The first (second) light exit surface preferably has a Lambertian emission profile. However, other more directed emission profiles are also conceivable.

The first (second) light exit surface can extend along an outer surface of a three-dimensional object. For example, the first (second) light exit surface can extend at least partially or completely along the outer surface of one of the following imaginary geometric objects: cone, truncated cone, ball, ellipsoid, pyramid, hyperboloid, cuboid, cube, cylinder, L-shape. Rotationally symmetrical geometries are preferred, but geometries without rotational symmetry are not precluded. In the case of a three-dimensional configuration, the first (second) lighting unit can have a plurality of first (second) emission directions which are distributed three-dimensionally around a center of the lighting device and/or the first (second) lighting unit. In this case at least one of the plurality of first (second) emission directions extends in operation through the working plane (facial plane). The effective surface size for the user is revealed in this case by the projection of the three-dimensional light exit surface onto the facial plane of the user. It is also possible that the first (second) light exit surface extends in a planar manner, that is to say it is spanned by two vectors. The first (second) light exit surface can then, for example, be rectangular or elliptical. In the case of a planar configuration the first (second) lighting unit has all the first (second) emission directions of the first (second) lighting unit preferably in the same direction, that is to say parallel to one another.

The first (second) light in operation can then be emitted through the first (second) light exit surface. The second light exit surface can be configured alternatively or additionally as a reflecting and/or light-scattering surface. In particular, the second light exit surface can be arranged in such a way that in operation it is illuminated by a light source of the second lighting unit which is not immediately visible, in particular, for the user, wherein the light reflected and/or scattered on the second light exit surface has the described characteristics for the second light. As a result, in particular, dazzling of the user by the second light source can be avoided.

According to one embodiment of the illumination device, the second light exit surface is reflecting and/or light-scattering. For example, for this purpose the illumination device contains a diffuser and/or a housing and/or a reflecting and/or light-scattering coating.

For artistic reasons, further light exit surfaces can also be implemented with further emission directions. Thus the lamp can also have a symmetrical structure, so that it can be arranged on a desk, on the right-hand side or on the left-hand side as required. In this case it is possible that a part of the light is not emitted in the direction of the facial plane of the user. This light is not specified in the following description, since it has no significant effect on the non-visual system of the user.

The first emission direction and the second emission direction are in particular adjustable in such a way and/or extend in such a way relative to one another that in operation of the illumination device the first (second) emission direction extends through a working plane (facial plane) of the user. In addition, the illumination device can be installed and/or positioned relative to one another in such a way that in operation of the illumination device the first (second) emission direction extends through a working plane (facial plane) of the user.

The illumination device can be constructed in such a way that the first (second) emission direction can be changed by the user. In this case and in what follows, the terms “changeable” and “adjustable” always relate to a mechanical non-destructive change or adjustment, preferably without the use of a special tool or of a tool. For example, for this purpose the illumination device has a mechanical rotating and/or pivoting mechanism, by means of which the first (second) lighting unit is connected to the second (first) lighting unit or to a main body of the illumination device. The illumination device can preferably be installed and/or positioned in such a way that the required illumination of the working plane and of the facial plane can always be achieved. An adjustable first (second) emission direction enables a fine adjustment of the first (second) emission direction even after the installation or the positioning of the illumination device, so that the installation and/or the positioning of the illumination device can be facilitated.

The first lighting unit and the second lighting unit can merge into one another or can be separate from one another. Furthermore, it is possible that a control unit is integrated into the first (second) lighting unit. In particular, the control unit can be a computer.

According to a preferred embodiment of the illumination device the first light has a first color temperature and the second light has a second color temperature. The first color temperature can be set in particular in a range of at least 2700 K and at most 6500 K. Alternatively or in addition, a melanopic action factor of the second light can be set in particular in a range between 0.4 and 1.1. Particularly preferably, the first color temperature and the melanopic action factor of the second light can be set.

Furthermore, it is possible that the second color temperature can be set, in particular, in a range of at least 5000 K and/or at most 10000 K. For a more aesthetic visual appearance a second color temperature can also be possible, which is less than 5000 K. The melanopic action factor of the second light can be set for example by means of the second color temperature. The first and the second color temperature can preferably be set independently of one another. For example, for setting of the first (second) color temperature the first (second) lighting unit contains spectral color LEDs which emit different colors, in particular a so-called red-green-blue LED, and/or LEDs which emit white light of a different color temperature. The color temperature can be set by targeted control of the individual LEDs.

Alternatively or in addition, preferably additionally, a first (second) illumination intensity of the first (second) light can be set individually. For example, this can be achieved by pulse width modulation in an electronic driver.

According to an embodiment of the illumination device, the first emission direction and the second emission direction can be set in such a way, and/or extend in such a way relative to one another in such a way, that the first emission direction and the second emission direction enclose an angle with one another of at least 50° and at most 130°, preferably at least 70° and at most 110°. Thus the first and the second emission direction extend transversely, in particular perpendicularly, relative to one another. Preferably the first emission direction in operation of the illumination device is directed downwards and the second emission direction is directed to the side.

According to one embodiment of the illumination device, at a spacing of at least 20 cm and at most 40 cm from the second lighting unit, the second light has a melanopic equivalent daylight (D65) illuminance (MEDI) of at least 240 lux. The melanopic equivalent daylight illuminance is a measurement for description of light with regard to its melanopic effects and is defined in the DIN SPEC 5031-100:2015 and/or the CEN TR 16791:2017. The melanopic equivalent daylight illuminance of the second light is preferably at least 240 lux according to PD CEN/TR 16791:2017. This corresponds to the value for the melanopic equivalent daylight (D65) illuminance. The second light can for example be cold white light, in particular of a LED, with a second color temperature of 6500 K, wherein with an illumination intensity of 300 lux in this case the required melanopic equivalent daylight illuminance of at least 240 lux can be achieved.

According to an embodiment of the illumination device the first lighting unit has a first light exit surface, on which and/or through which the first light is emitted in operation of the illumination device, wherein the first light exit surface in operation has a first light density of at most 60,000 cd/m², in particular at most 10,000 cd/m². Alternatively or in addition, the second lighting unit has a second light exit surface, on which and/or through which the second light is emitted in operation of the illumination device, wherein the second light exit surface in operation has a second light density of at most 3000 cd/m². The maximum first light density corresponds in particular to the “free group” with regard to photochemical retinal damage according to the standard DIN EN 62471 (version at the date of filing). In particular, the second light density averaged spatially over the light exit surface is at most 1000 cd/m² and the maximum second light density is at most 3000 cd/m². As a result the illumination device can also be used at computer workstations according to the standard EN 12464-1 (version at the date of filing).

According to an embodiment of the illumination device, the first light has a first luminous flux of at least 100 lumen, in particular at least 140 lumen. The first luminous flux is preferably at most 250 lumen, particularly preferably at most 200 lumen. This is advantageous in particular in the case of a working plane of the size DIN A3. For the illumination of a working plane of this size with a first illumination intensity of 500 lux, a first luminous flux of for instance 60 lumen in the central area of the working plane is required in particular. Since the first illumination intensity outside a central surface of the working plane advantageously declines, not abruptly but gradually, a broader emission is preferable, resulting in an additional luminous flux requirement of for instance 60 lumen to 140 lumen. The first lighting unit preferably emits a first luminous flux of at least 100 lumen and at most 200 lumen. In the case of an emitted first luminous flux of 200 lumen, the size of the first light exit surface is preferably at least 60 cm². In particular, the size of the first light exit surface can preferably be at most 70 cm². In this way it can be guaranteed that the first light density is at most 10,000 cd/m².

The second light exit surface is preferably larger than the first light exit surface. As a result, in particular, it is possible to compensate for the movement of the user. For example, the second light exit surface is a rectangle with a width of at least 15 cm, preferably at least 30 cm, and a height of at least 10 cm, preferably at least 20 cm. The second light exit surface is in particular at least 300 cm² and at most 700 cm², preferably 500 cm². Furthermore, due to a large second light exit surface it can be ensured that a maximum second light density of 3000 cd/m² is not exceeded.

The first illumination intensity for the illumination of a desk for office work is in particular 500 lux (standard EN 12464). Other first illumination intensities can also be implemented for other work environments. For example, the first illumination intensity for precision work is 1000 lux or for precise assembly or adjustment work is 1500 lux. The luminous flux and the size of the light exit surface should be adapted as required to other work environments.

According to an embodiment of the illumination device, a first color rendering index (CRI) of the first light is at least 80, in particular at least 90. In particular this meets the requirements of the standard EN 12464 (version at the date of filing). Alternatively or in addition, a second color rendering index of the second light is at least 70, in particular at least 80. Since the second light does not serve for illumination of the working plane, there is no particular requirement for color quality according to EN 12464. Since the light illuminates the facial part of the user, however, the color rendering index should have a value which still allows facial colors with a natural appearance (at least 70). Alternatively or in addition, a flicker index of the second light is at most 0.25, in particular at most 0.15 for frequencies below 100 Hz. The flicker index is in particular the flicker index according to IESNA (version at the date of filing; also: CIE TN-006:2016, in particular page 9). Alternatively or in addition, the degree of flickering (short term flicker indicator) P_{ST} according to IEC TR 61547-4-15 (version at the date of filing) is less than 1.5.

According to an embodiment, the illumination device can be installed and/or positioned in such a way that a first spacing of the first lighting unit relative to the working plane and/or a second spacing of the second lighting unit relative to the facial plane are/is changeable. For example, the illumination device can be adjustable in height and/or laterally.

According to an embodiment, the first (second) lighting unit is mounted in the illumination device in such a way that the first (second) emission direction of the first (second) lighting unit can be adjusted individually by the user. The first (second) lighting unit can, for example, be mounted tiltably, pivotably and/or adjustable laterally and/or in height.

According to an embodiment, the first lighting unit and/or the second lighting unit can be coupled to a control unit, wherein the first color temperature, the second color temperature, a first illumination intensity of the first light and/or a second illumination intensity of the second light can be set by means of the control unit. The control unit is preferably configured in order to activate the first (second) lighting unit electrically, in particular in such a way that the first (second) lighting unit can fulfil illuminating functions. “Illuminating functions” can be understood to mean for example a melanopic effect and/or an illumination effect, in particular for illuminating a working area.

The first and the second lighting unit, in particular the illuminating functions thereof, can preferably be activated independently of one another by means of the control unit. Furthermore, it is possible that the first and the second lighting unit, in particular the illuminating functions thereof, can be coordinated with one another by means of the control unit. In other words, settings of the first lighting unit can be dependent upon settings of the second lighting unit and/or vice versa.

According to an embodiment, the illumination device has an, in particular wireless, connecting unit. The control unit is connectable by means of the connecting unit to the illumination device. The control unit can have a further, in particular wireless, connecting unit, by means of which a connection can be guaranteed. A wireless connection can be provided for example by means of Bluetooth, WLAN, Zigbee, Li-Fi and/or by means of an infrared connection. Other transmission techniques are also conceivable.

Furthermore, a control unit is specified. The control unit is preferably configured for operation with a previously described illumination device. In other words, all features disclosed in connection with the illumination device are also disclosed for the control unit, and vice versa.

According to an embodiment, the control unit is configured for setting the first color temperature, the first illumination intensity, the second color temperature and/or the second illumination intensity.

For setting, the control unit can, in particular, have an operating element by means of which the user can interact with the control unit, and thus with the first (second) lighting unit. The operating element can be an operating panel, a user interface, a switch and/or a push button. Furthermore, the control unit can contain components which as a function of the operation by a user and/or of external influences, such as for example data from a sensor, the clock time and/or the position of the control unit, perform a predetermined illuminating function of the first (second) lighting unit. The activation, that is to say the setting, can take place in a simple case by switching on or off of the first (second) lighting unit. However, more complex activation variants can be possible, in which different illuminating functions for the first (second) lighting unit can be activated. For example, the illuminating functions can be activated as a function of a clock time. It is possible that the activation can be changed dynamically by the user. Alternatively or in addition, the activation can also take place by means of an automated system, which can be integrated into the control unit or which can be coupled to the control unit.

According to an embodiment of the control unit, the control unit is a portable computer, in particular a smartphone or a tablet. Alternatively it is possible that the control unit can be coupled to a portable computer, for example by means of a wireless connection. For this purpose the control unit can have the further connecting unit and can be integrated into the illumination device and in particular directly, that is to say by means of a wired connection, and connected to the first and the second lighting unit.

Furthermore, a method for operating an illumination device is specified. The method can preferably be carried out with an illumination device described here, particularly preferably using a control unit described here, or the illumination device and/or the control unit are/is preferably configured in order to be operated by the method. This means that all features disclosed in connection with the illumination device and the control unit are also disclosed for the method, and vice versa.

The method comprises the following steps:
defining a first control curve according to a diurnal working rhythm,
defining a second control curve according to a circadian rhythm,
setting and/or changing the first color temperature and/or the first illumination intensity of the first lighting unit according to the first control curve,
setting and/or changing the second color temperature and/or the second illumination intensity of the second lighting unit according to the second control curve.

The first color temperature, the first illumination intensity, the second color temperature and/or the second color temperature are generally parameters of the first light and/or the second light.

The method is, particularly preferably, not a therapeutic method or a therapeutic treatment. In fact, the method preferably relates exclusively to the operation of the illumination device. The light emitted by the illumination device during operation, in particular second light emitted by the second lighting unit, can be used for example in particular for non-therapeutic improvement of performance. The first and the second control curve preferably differ.

The first (second) control curve can be saved in the illumination device and/or the control unit. The activation of the illumination device can then take place automatically with the aid of the stored first (second) control curve.

According to an embodiment, the first (second) control curve is a function of at least one of the following parameters: time of day, season, sunrise time, sunset time, working time. In particular the first control curve can be adapted to the working time, whilst the second control curve can be adapted to the biological diurnal rhythm.

It is possible to adapt the first (second) control curve to the sunrise and/or the sunset time. For example, the second control curve can be configured in such a way that during a time interval, for example for half an hour before sunrise, the second color temperature of the second light is changed gradually from warm white (i.e. a color temperature of at most 3200 K) into cold white (i.e. a color temperature of at least 5000 K), so that chronologically shifted after sunrise, for example half an hour after sunrise, the second light is cold white. In this case the cold white proportion of the second light can be gradually increased and the warm white proportion of the second light can be gradually reduced, so that the total emitted white light initially contains substantially only the warm white mixed light (with a low melanopic action factor) and towards the end contains substantially only the cold white mixed light (with a high melanopic action factor). Alternatively or in addition, at sunset the same gradual change can take place, but in the opposite direction. Analogously an activation in the event of the first lighting unit can take place by means of the second control curve, wherein the alteration of the first color temperature of the first light takes place at the time of starting work. Furthermore, an analogous activation of the first (second) illumination intensity can take place, wherein the first (second) illumination intensity is increased in the morning and reduced in the evening.

It is possible that the illumination device has an external light sensor and/or is connected to an external light sensor. In this case the configuration of the first (second) control curve can also be set by means of the daylight actually present instead of or in addition to the clock time.

### BRIEF DESCRIPTION OF THE DRAWINGS

Preferred further embodiments of the invention are explained in greater detail by the following description of the drawings.

FIGS. 1, 2A, 2B, 2C, 2D, 2E, 2F, 2G, 2H, 2I and 2J show exemplary embodiments of an illumination device described here.

FIGS. 3A and 3B show exemplary embodiments of a method described here.

FIG. 4 shows an exemplary embodiment of a control unit described here.

### DETAILED DESCRIPTION OF THE DRAWINGS

The illumination device described here, the control unit described here as well as the method described here are explained in greater detail below with reference to exemplary embodiments and the associated drawings. In this case elements which are the same, of the same kind, similar or equivalent are provided with the same reference numerals. Repeated description of some of these elements is omitted in order to avoid redundancies.

The drawings and the size ratios of the elements illustrated in the drawings elements should not be regarded as drawn to scale relative to one another. On the contrary, individual elements may be shown as excessively large for better illustration and/or to aid understanding.

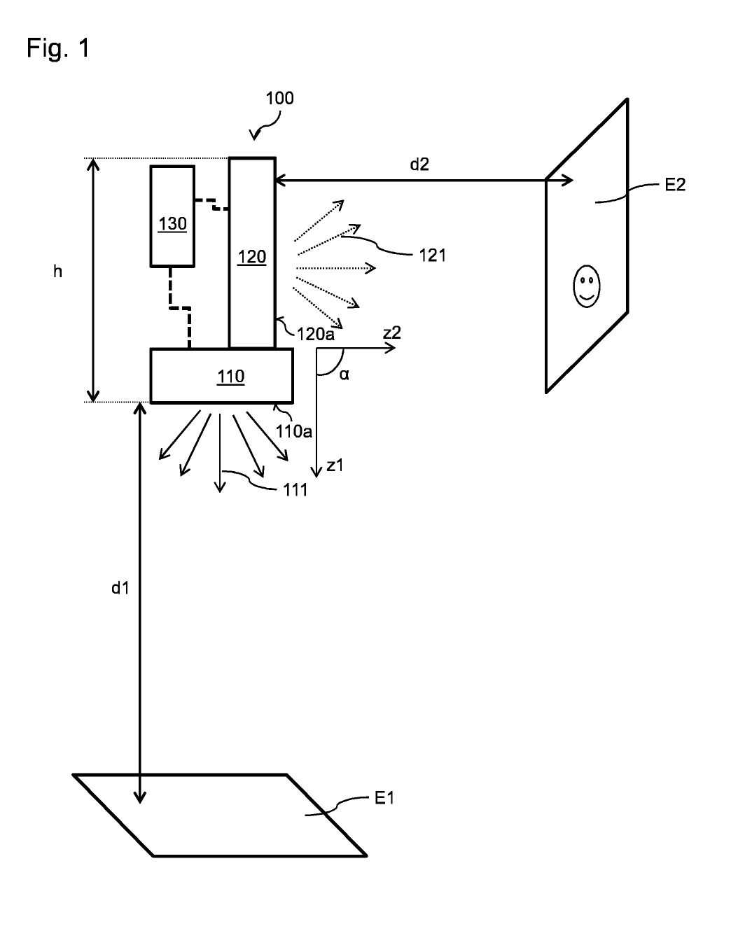

An exemplary embodiment of an illumination device 100 described here is explained in greater detail with reference to the schematic sectional view in FIG. 1. The illumination device 100 comprises a first lighting unit 110, which in operation emits a first light 111 to and/or through a first light exit surface 110a in a first emission direction z1, and a second lighting unit 120, which in operation emits a second light 121 to and/or through a second light exit surface 120a in a second emission direction z2. The first emission direction z1 and the second emission direction z2 enclose an angle α which is at least 50° and at most 130°, in the present exemplary embodiment in particular 90°. The first lighting unit 110 and the second lighting unit 120 can in each case be connected to a control unit 130.

Generally the first (second) lighting unit 110 (120) can be made up of a plurality of individual parts, in particular a plurality of first (second) light exit surfaces 110a (120a). Each of the individual parts by itself can meet any possible requirements relating to maximum first (second) light density and first (second) color temperature. The first (second) lighting unit 110 (120) as a whole preferably meets any requirements relating to the first (second) illumination intensity.

The illumination device 100 can have further lighting units (not shown in the drawings) which in operation emit a further light in spatial directions which do not extend through the facial plane E1 or the working plane E2. This further light can have any quality and is in particular not relevant for the described invention. However, there may be artistic or other reasons for emitting further light, which is not necessary either for the visual effect of the first light 111 or for the non-visual effect of the second light 121.

The illumination device 100 is in particular mounted in such a way that the first emission direction z1 extends through a working plane E1 and the second emission direction z2 through a facial plane E2. The working plane E1 can have a visual task area and can in particular be part of a desk. The facial plane E2 extends in particular through a face, preferably the eyes, of a user.

The illumination device 100 can be designed as a ceiling light or as a table lamp. For example the illumination device 100 contains yet further mounting components (not shown in the drawings), such as a lamp base (i.e., a supporting foot of a standard lamp) and/or a suspension device. By means of such components it is possible in particular to set a position of the illumination device 100 with respect to the working plane E1 and/or the facial plane E2.

A first spacing d1 between the first light exit surface 110a and the working plane E1 is for example at least 30 cm and at most 50 cm, preferably at most 35 cm. A second spacing d2 between the second light exit surface 120a and the working plane E2 is for example at least 30 cm and at most 50 cm, preferably at most 35 cm. A height h of the illumination device 100 corresponds to a spacing of the first light exit surface 110a from an upper edge of the second lighting unit 120. For example, the height h is at least 20 cm and at most 50 cm, in particular 25 cm. An extent of the second light exit surface 120a along the height h preferably corresponds to at least 80%, particularly preferably at least 90%, of the height h.

The working plane E1 can be located in particular at table height, that is to say for example at a height of at least 70 cm and at most 95 cm above a base. For example, in this case the first light exit surface 110a is located at least 100 cm and at most 130 cm above the base, in particular 35 cm above the working plane E1. A center of the second light exit surface 120a is located preferably 40 cm above the working plane E1, in particular at a height of at least 110 cm and at most 135 cm above the base.

The first spacing d1 and/or the second spacing d2 can be adjustable in each case. In other words, the height and/or the lateral position of the illumination device 100 can be variable.

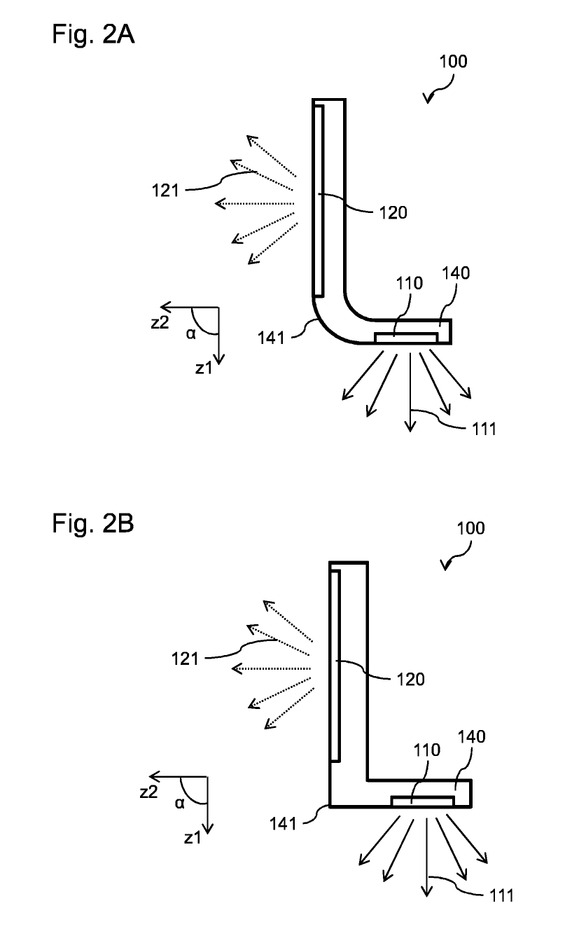

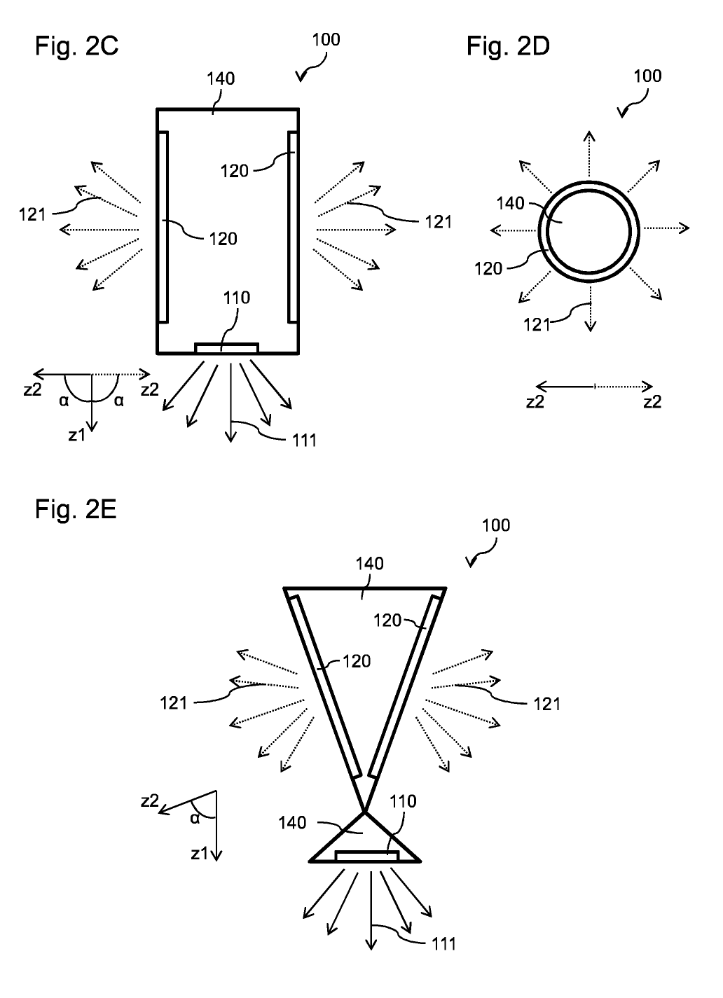

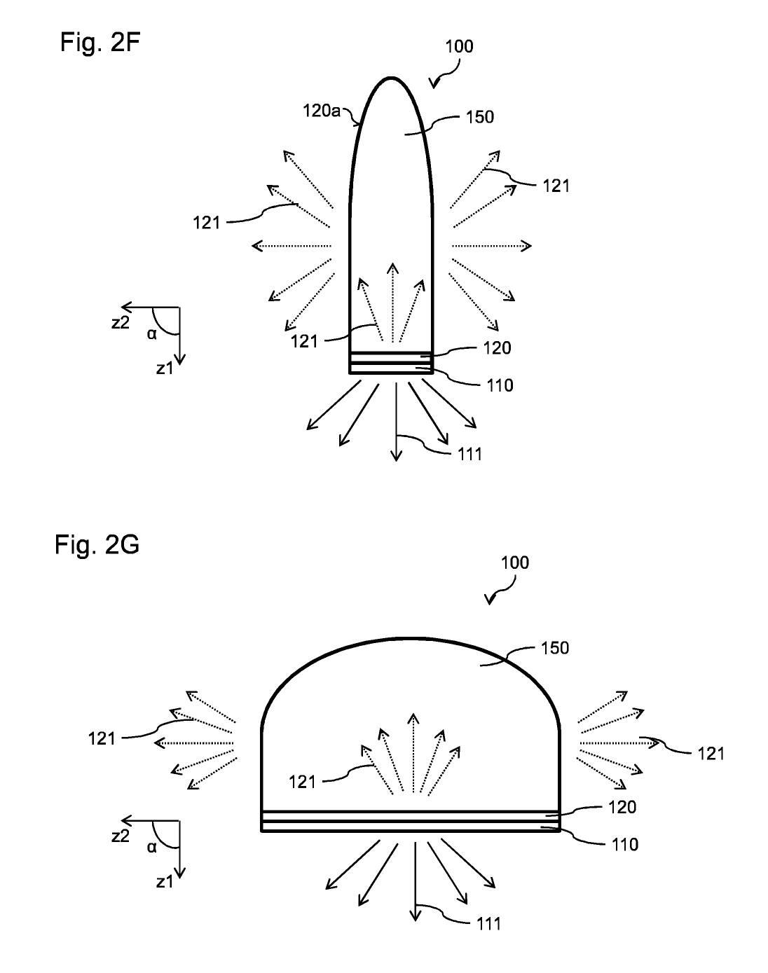

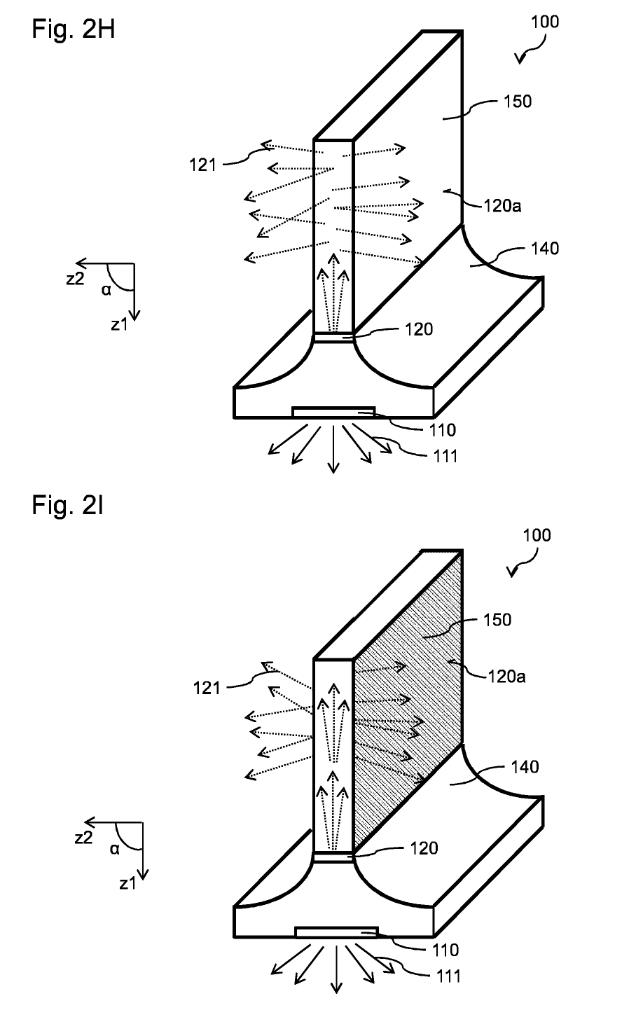

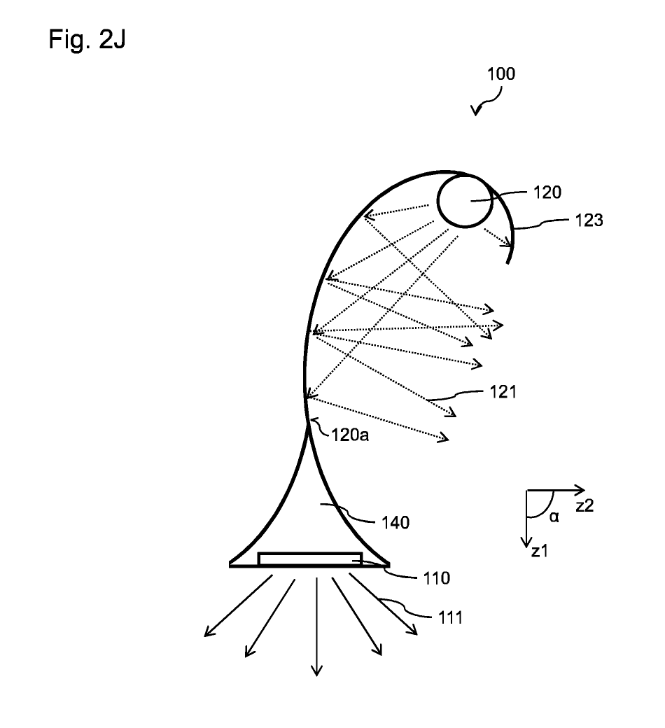

With reference to the schematic sectional representations of FIGS. 2A, 2B, 2C, 2E, 2F, 2G and 2J and the schematic plan views of FIGS. 2D, 2H and 2I exemplary embodiments of an illumination device described here are explained in greater detail. The illumination device 100 according to the exemplary embodiments of FIGS. 2A to 2E in each case have a housing 140, on which or into which the first lighting unit 110 and the second lighting unit 120 are mounted or introduced. In the exemplary embodiments FIGS. 2F, 2G, 2H and 2J the second lighting unit 120 has, alternatively or in addition, a diffuser 150. In the exemplary embodiment of FIG. 2J the second light exit surface 120a of the second lighting unit 120 is designed to be reflecting.

The illumination device 100 according to the exemplary embodiments of FIGS. 2A, 2B and 2J are in each case asymmetric. In other words, the second lighting unit 120 is arranged on one side. In the exemplary embodiments of FIGS. 2A and 2B the housing 140 is L-shaped, wherein an outer edge 141 of the housing 140 in the exemplary embodiment of the FIG. 2A is rounded and in the exemplary embodiment of FIG. 2B extends at an angle. In general the illumination device 100 can be in particular mounted in such a way that the second emission direction z2 extends through a facial plane E2 of the user and the first emission direction z1 extends through a working plane E1. An asymmetric arrangement can be in particular advantageous if only the facial plane E2 of one single user should be illuminated.

The illumination devices 100 according to the exemplary embodiments of FIGS. 2C to 2I are in each case asymmetric. In particular, the illumination devices 100 of FIGS. 2C to 2G are rotationally symmetrical, preferably along an axis of rotation extending parallel to the first emission direction z1.

The second lighting unit 120 can be arranged rotationally symmetrical around the housing 140 (cf. exemplary embodiments of FIGS. 2C to 2E). The housing 140 can have the shape of a cylinder, a cuboid, a pyramid or a cone. In the exemplary embodiment FIG. 2E the housing 140 comprises in particular two cones, of which the tips meet. A symmetrical arrangement of the illumination device 100 is in particular advantageous when the illumination device 100 has a lamp base. Thus an arrangement of the illumination device 100 can take place on any side of the user and/or facial planes E2 of a plurality of users can be illuminated.

In general during operation, the first light 111 and/or second light 121 can be emitted along a plurality of first emission directions z1 and/or second emission directions z2. This is the case in particular in the event of a rotationally symmetrical arrangement, but also for example in the case of a flat emitter as first lighting unit 110 and/or as second lighting unit 120. At least one of the first emission directions z1 then extends through the working plane E1 and at least one of the second emission directions z2 extends through the facial plane E2. Furthermore, it is possible that one of the second emission directions z2 extends through a first facial plane E2 and a further one of the second emission directions z2 extends through a second facial plane E2. In other words, the non-visual effect of the second light 121 can act on a plurality of users.

In the exemplary embodiments FIGS. 2F, 2G, 2H and 2I the second lighting unit 120 has in each case a diffuser 150. The second lighting unit 120 is mounted above the first lighting unit 110. A lighting unit of the second lighting unit 120 faces in operation the second light 121 upwardly. In the exemplary embodiments the drawings 2F, 2G and 2H the diffuser 150 is in particular light-scattering and/or translucent, so that the second light 121 is scattered and laterally deflected inside the diffuser 150 and subsequently is propagated along the second emission direction z2. However, the diffuser 150 can also be formed with a transparent material. For example, the diffuser 150 then contains diffuser particles and/or a coating (cf. FIG. 2I), by means of which a scattering in the second emission direction z2 can be implemented.

As illustrated in FIG. 2I, the diffuser 150 can have on its outer surface a light-scattering and/or reflecting coating (shown cross-hatched in FIG. 2I). In this case the diffuser 150 can be transparent. However, other configurations are possible. The light emitted by the second lighting unit 120 can propagate through the diffuser 150 and can be deflected on the outer surfaces thereof in the second emission direction z2.

The diffuser 150 can be formed with a plastic material or can be made of such a material. For example, the diffuser 150 is formed with at least one of the following materials: acrylic, polycarbonate, polymethyl methacrylate (also referred to as Plexiglas). Furthermore, the diffuser 150 can in particular contain reflecting microparticles which enable a scattering of light. Alternatively or in addition, the diffuser 150 can have other structures which can change the index of refraction, in particular alterations in the structure of the material of the diffuser 150. In the case of a light-scattering coating the coating can be formed with microparticles and/or other light-reflecting structures.

It is in particular possible that the diffuser 150 has characteristics of a waveguide. The diffuser 150 can appear transparent when the illumination device 100 is switched off. This enables an aesthetic and light visual appearance for the user. Alternatively it is possible that an outer surface of the diffuser 150 has a white and/or reflecting coating and the outer surface of the diffuser 150 facing the user is light-permeable. The emission then takes place on one side. The coating can also contain graphic elements as signs, images or the like.

FIG. 2J shows an exemplary embodiment of the illumination device 100, in which the second light exit surface 120a is designed to be reflecting. The second lighting unit 120 is arranged in such a way that its emission takes place in the direction of the second light exit surface 120a. Light reflected on the second light exit surface 120a is emitted as second light 121 along the second emission direction z2. Furthermore, the illumination device 100 can have a diaphragm 123 which is configured in order to block emitted light directly by the second lighting unit 120 in the direction of the facial plane E2, that is to say in the second emission direction z2, so that dazzling of the user by direct light can be avoided. In this exemplary embodiment the second light exit surface 120a is preferably at least partially curved. Due to a reflecting second light exit surface 120a the illumination device 100 can act as a flat light source.

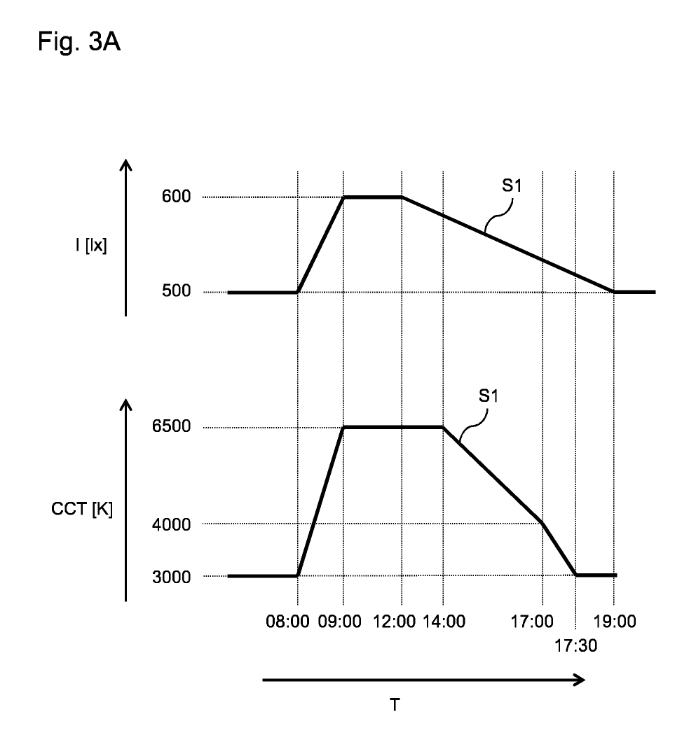

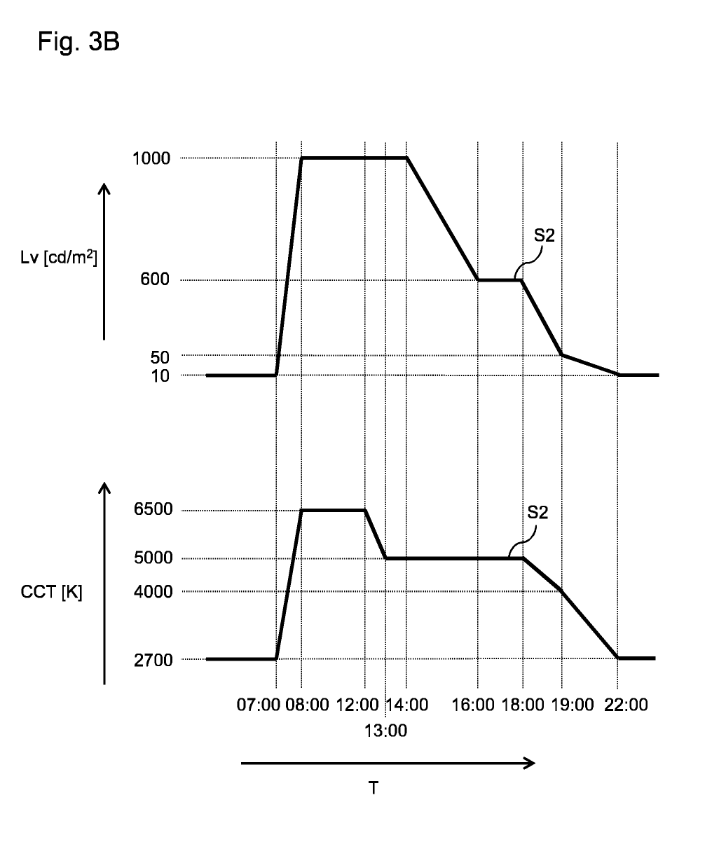

FIGS. 3A and 3B show exemplary embodiments of a method for operating an illumination device. In the method, variably in the daytime, parameters of the first light 111 (FIG. 3A) are set according to a first control curve S1 and parameters of the second light 121 (FIG. 3B) are set according to a second control curve S2. The parameters are, purely by way of example, the first illumination intensity of the first light 111 (FIG. 3A at the top, intensity in lux), the first color temperature of the first light (FIG. 3A at the bottom, CCT in K), the second light density of the second light 121 (FIG. 3B at the top, light density in cd/m²) and the second color temperature of the second light 121 (FIG. 2C at the bottom, CCT in K).

The first control curve S1 (FIG. 3A) corresponds to the illumination of the working plane E1 with the first light 111. The first illumination intensity is increased from 8 a.m. to 9 a.m. according to a predetermined scheme from 500 lux to 600 lux. This level is maintained until 12 noon and then reduced again steadily to 500 lux by 7 p.m. Between 7 p.m. and 6 a.m. the first illumination intensity remains at 500 lux.

Simultaneously with the change of the illumination intensity described above, the first color temperature increases between 8 a.m. and 9 a.m. von 3000 K to 6500 K, is maintained at 6500 K until 2 p.m., drops to 4000 K by 5 p.m. and then goes down to 3000 K within 30 minutes (by 5.30 p.m.). Between 5.30 p.m. and 8 a.m. the first color temperature is 3000 K.

After being switched off and switched on again, the light emission of the first lighting unit 110 can be set to the appropriate value, depending upon the dynamics described above, according to the first control curve S1.

The second control curve S2 (FIG. 3B) corresponds to the illumination of the facial plane E2. The second light density of the second light 121 increases from 7 a.m. to 8 a.m. from 10 cd/m² to 1000 cd/m² and maintains this value until 2 p.m. From 2 p.m. to 4 p.m. the second light density goes down to 600 cd/m². The value is maintained until 6 p.m. and then goes down by 7 p.m. to 50 cd/m². From 10 p.m. the second light density goes back down to 10 cd/m². This value is maintained between 10 p.m. and 7 a.m. It is possible that some of these values, in particular the last value, achieve no biological effect on the user, but merely have a decorative or emotional effect. The objective is to minimize the disturbing effect of the second light 121 on the non-visual system of the user during the evening.

Simultaneously with the change to the second light density, the second color temperature of the second light 121 increases between 7 a.m. and 8 a.m. from 2700 K to 6500 K, maintains this value until 12 noon, goes down within the next hour (by 1 p.m.) to 5000 K and maintains this value until 6 p.m. From 6 p.m. to 7 p.m. the second color temperature goes down to 4000 K, and by 10 p.m. it goes down to 2700 K and maintains this value until 7 a.m. the next morning.

After being switched off and switched on again, the light emission of the second lighting unit 110 can be set to the appropriate value, depending upon the dynamics described above, according to the second control curve S2.

The chronological sequence described in connection with FIGS. 3A and 3B corresponds to a working cycle for a person, who wishes to work in a concentrated manner between 8 a.m. and 5 p.m., only occasionally before 8 a.m. and after 5 p.m. and not with the objective of having particularly good concentration and being alert, and before 6 a.m. and after 7 p.m. uses the illumination primarily for relaxing activities.

The configuration of the first control curve S1 and the second control curve S2 described above should be understood to be purely exemplary. The activation of the first lighting unit 110 can take place according to the first control curve S1 and the activation of the second lighting unit 120 can take place, in particular independently of the first lighting unit 110, according to the second control curve S2. The parameters of the first control curve S1 and/or the second control curve S2 can be changed by the user, for example by means of a control unit 130.

According to a first setting, the first lighting unit 110 and the second lighting unit 120 can be activated in such a way that the first lighting unit generates the first light 111 with a first illumination intensity of 550 lux at a first color temperature of 4000 K on a table surface, and the second lighting unit 120 generates the second light 121 with a second illumination intensity of 350 lux and cold white light with a second color temperature of 6500 K. This can correspond to a melanopic daylight-equivalent illumination intensity of 280 lux, in particular affecting the eye of the user, preferably in a second spacing d2 of the facial plane E2 from the second light exit surface 120a of at least 30 cm and at most 40 cm. The working plane E1 can extend through the table surface and the facial plane E2 can extend through the eye. This first setting corresponds to a static work setting for use in the daytime with a standard (EN 12464) illumination of the working surface and a non-visually effective illumination of the user.

According to a second setting, the first lighting unit 110 and the second lighting unit 120 can be activated in such a way that the first lighting unit generates the first light 111 with a first illumination intensity of 300 lux at a first color temperature of 2700 K on the working plane E1, and the second lighting unit 120 generates the second light 121 with a second illumination intensity of at most 50 lux and cold white light with a second color temperature of 2500 K. This second setting would be suitable for activities which are visually not very demanding in the evening or at night and avoids stimulation of the non-visual system.

It is possible that the user can alternate between the first setting and the second setting, and in particular can set gradual intermediate values, by means of a setting function, for example on the control unit 130. The setting of these intermediate values can also take place dynamically by an automatic time control. Such an automatic time control can then correspond to the method described here, in which parameters of the first light 111 and/or of the second light 121 discontinued are set.

The setting of the parameters of the first light 111 and/or of the second light 121 can be influenced by means of a sensor. For example, the sensor is an ambient light sensor and/or a motion sensor. The sensor can for example detect the presence of persons, so that the illumination is switched off when no person is located in the range of the illumination device 100. Furthermore, the sensor can determine the overall brightness in the environment of the illumination device 100 and for example, if sufficient daylight is available, it can reduce the emitted luminous flux from the first lighting unit 110 and/or the second lighting unit 120.

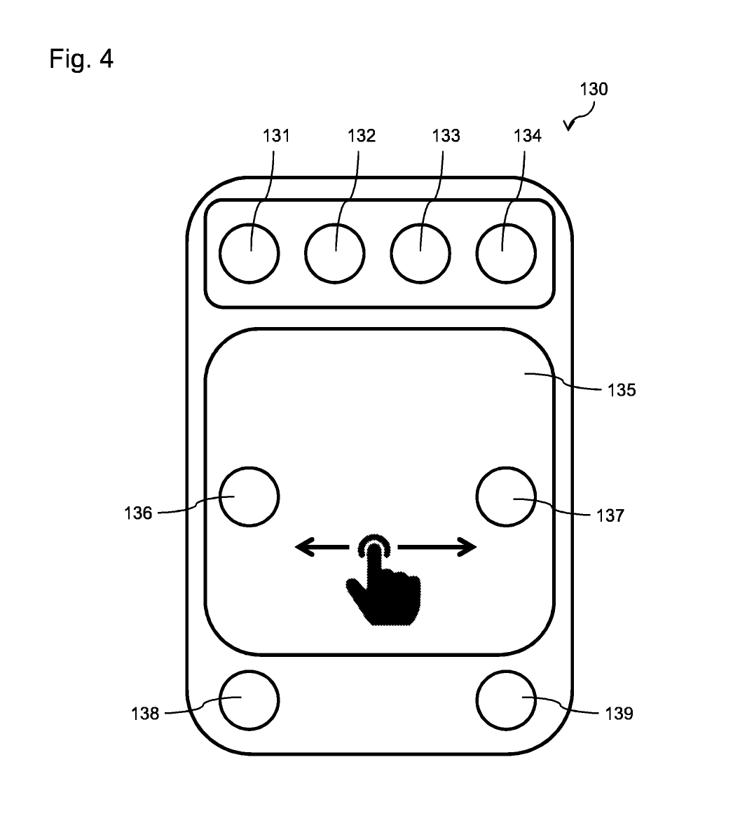

An exemplary embodiment of a control unit 130 described here is explained in greater detail with reference to the schematic representation in FIG. 4. This shows an operating panel of a user interface of the control unit 130. The operating panel has different selection possibilities using operating areas 131-139. The control unit 100 can have any combination of the operating areas 131-139. The operating areas 131-139 can be configured, in particular, as icons on a touchpad. The operating areas 131-139 can also be implemented as a user interface of a computer program, in particular, an application on a mobile computer, or on a remote control.

A first to fourth operating area 131-134 can in each case correspond to a pre-defined setting for illuminating functions, between which the user can choose. For example, the first operating area 131 corresponds to a setting for working at night (for example 500 lux as first illumination intensity, 3000 K as first color temperature, 10 lux as second illumination intensity and 2700 K as second color temperature). The second operating area 132 corresponds to a setting for relaxing (for example 50 lux as first illumination intensity, 3000 K as first color temperature, 2-3 lux as second illumination intensity and 5600 K as second color temperature). The third operating area 133 can correspond to a setting for concentrated work (for example 500 lux as first illumination intensity, 4000 K as first color temperature, 300 lux as second illumination intensity and 6500 K as second color temperature). The fourth operating area 134 can correspond to a setting for working during the day (for example 500 lux as first illumination intensity, 4000 K as first color temperature, 200 lux as second illumination intensity and 5000 K as second color temperature).

By means of a fifth operating area 135 a selection can be made for example between an automatic operation, in particular corresponding to a first control curve S1 and/or a second control curve S2, or a manual operation by means of user inputs. The automatic operation can correspond to a gradual change between the settings according to the first to fourth operating area 131-134. The automatic operation can be selected as standard when switching on the illumination device 100.

A sixth operating area 136 and a seventh operating area 137 can correspond to a setting in which a selection can be made between two extremes, for example between a setting for night time and a setting for day time. In the manual operation, a selection can be made, for example, gradually between these two settings. The manual operation can, for example, be prevented at specific times of day by means of a timer, in order for example to avoid undesirably high illumination intensities at night.

By means of the eighth operating area 138, a so-called sleep function can for example be started, in which the illumination device 100 switches itself off after a predetermined time, for example 15 minutes. The ninth operating area 139 can correspond to an on/off switch.

The invention is not limited to these embodiments by the description with reference to the exemplary embodiments. On the contrary, the invention encompasses each new feature as well as any combination of features, in particular including any combination of features in the claims, even if this feature or this combination itself is not explicitly given in the claims or the exemplary embodiments.

1. An illumination device comprising:
a first lighting unit that emits a first light in a first emission direction, the first emission direction extends through a working plane of a user; and
a second lighting unit that emits a second light in a second emission direction, the second emission direction extends through a facial plane of the user.

2. The illumination device of claim 1, the first light has a first color temperature, the second light has a melanopic action factor, wherein the first color temperature can be set in a range of at least 2700 K and at most 6500 K, and the melanopic action factor of the second light can be set between 0.4 and 1.1.

3. The illumination device of claim 1, wherein the first emission direction and the second emission direction enclose an angle of at least 50° and at most 130°.

4. The illumination device of claim 1, wherein a melanopic equivalent daylight (D65) illuminance (MEDI) of the second light is at least 240 lux at a spacing of at least 20 cm and at most 40 cm from the second lighting unit.

5. The illumination device of claim 1, wherein the first lighting unit has a first light exit surface, the first light is emitted in operation of the illumination device from the first light exit surface, wherein the first light exit surface in operation has a first light density of at most 60,000 cd/m².

6. The illumination device of claim 5, wherein the first light density is at most 10,000 cd/m².

7. The illumination device of claim 1, wherein the second lighting unit has a second light exit surface, the second light is emitted in operation of the illumination device from the second light exit surface, wherein the second light exit surface in operation has a second light density of at most 3000 cd/m².

8. The illumination device of claim 7, wherein the second light exit surface is at least one of light scattering or reflective.

9. The illumination device of claim 1, wherein the first light has a first luminous flux of at least 100 lumen.

10. The illumination device of claim 9, wherein the first luminous flux is at least 140 lumen.

11. The illumination device of claim 1, further comprising:
a first color rendering index of the first light of at least 80;
a second color rendering index of the second light of at least 70;
a flicker index of the second light of at most 0.25; and
a degree of flickering P_{ST} of less than 1.5.

12. The illumination device of claim 1, the illumination device having a first spacing of the first lighting unit relative to the working plane and a second spacing of the second lighting unit relative to the facial plane, the first spacing and the second spacing are changeable.

13. The illumination device of claim 1, wherein the first lighting unit and the second lighting unit is adjustably mounted to a mounting component, the first emission direction of the first lighting unit and the second emission direction of the second lighting unit is user adjustable.

14. The illumination device of claim 1, wherein the first lighting unit and the second lighting unit is coupled to a control unit, wherein the control unit sets at least one parameter from the group consisting of a first color temperature, a second color temperature, a first illumination intensity of the first light, and a second illumination intensity of the second light.

15. The illumination device of claim 1, further comprising a connecting unit, the control unit configured to be in connection to the first lighting unit and second lighting unit by the connecting unit.

16. The illumination device of claim 15, wherein the connecting unit utilizes a wireless connection protocol.

17. A control unit for an illumination device according to claim 1, the control unit comprising:
an operating element, the operating element configured to set a property selected from the group consisting of the first color temperature, the first illumination intensity, the second color temperature, and the second illumination intensity.

18. A control unit according to claim 17, wherein the control unit is a portable computer, smartphone, or a tablet.

19. A method for operating an illumination device according to claim 1, the method comprising the following steps:
defining a first control curve according to a diurnal working rhythm;
defining a second control curve according to a circadian rhythm;
setting at least one property according to the first control curve, the at least one property according to the first control curve selected from the group consisting of a first color temperature and a first illumination intensity of the first lighting unit; and
setting at least one property according to the second control curve, the at least one property according to the second control curve selected from the group consisting of a second color temperature and a second illumination intensity of the second lighting unit.
